(19) United States
(12) Patent Application Publication
(10) Pub. No.: US 2019/0015636 A1
(43) Pub. Date: Jan. 17, 2019

(54) Medical Component Securement Device

(71) Applicant: John Robinson, Chambersburg, PA (US)

(72) Inventor: John Robinson, Chambersburg, PA (US)

(21) Appl. No.: 16/136,798

(22) Filed: Sep. 20, 2018

Related U.S. Application Data
(63) Continuation of application No. PCT/US17/23166, filed on Mar. 20, 2017.
(60) Provisional application No. 62/311,967, filed on Mar. 23, 2016.

(51) Int. Cl. A61M 25/02

(52) U.S. Cl. CPC A61M 25/02; A61M 2025/0206; A61M 2025/028

(57) ABSTRACT

A medical component securement device includes a strap and a first medical component holder attached to the strap. The strap includes a number of openings spaced along the length of the strap that enables one or more auxiliary medical component holders to be removably attached to the strap. The first medical component holder may include two members that pivot about a pivot axis to open and close the first medical component holder.

### RELATED APPLICATION

This application is a continuation of and claims priority from my co-pending PCT application PCT/US17/23166 “Medical Component Securement Device” filed Mar. 20, 2017 at Attorney Docket No. 1-2283-PCT, which in turn claims priority from my now expired U.S. Provisional Patent Application No. 62/311,967 “Intravenous Device” filed Mar. 23, 2016 at Attorney Docket No. 1-2283-P, which priority applications are incorporated by reference as if fully set forth herein.

### FIELD OF THE DISCLOSURE

The disclosure relates generally to devices for securing medical components to a patient's body, and more specifically, to devices for securing tubular medical components such as catheters or medical tubing to the patient's body.

### BACKGROUND OF THE DISCLOSURE

Medical treatment of a patient (human or other mammal) often includes insertion of a catheter or like tubular medical component into the patient's body for introduction or withdrawal of fluids. The catheter is connected to tubing that flows the liquid to or from the catheter.

Medical components such as catheters and the tubing attached to the catheters are often secured to the patient by applying adhesive tape over the component and adhering the tape to the patient's skin. Adhesive tape, however, may cause skin irritation and is often painful to remove.

Medical component securement devices that hold and secure medical components to the patient have been developed that eliminate the use of adhesive tape. One such securement device disclosed in Hasslinger, U.S. Pat. No. 4,569,348 includes a first strap that wraps around and holds the medical component, the first strap being attachable to a second strap that wraps around a body part and secures the first strap to the patient's body. Hook and loop fasteners secure the first strap around the medical component, secure the first strap to the second strap, and secure the ends of the second strap together to fasten the securement device to the body.

When holding and securing a medical component to the patient using the straps, the hook and loop fasteners may inadvertently contact and adhere to one another before the first strap is properly wrapped around the medical component or before the second strap is properly wrapped about the patient. Re-adjustment and re-positioning of the straps and the component held by the straps is often required before the component is suitably positioned and secured to the body.

Furthermore, the straps making up the holder are not rigid and are very flexible. A strap may buckle in use. Buckling of the strap causes the medical component to shift with respect to the patient's body.

Bierman U.S. Pat. No. 5,578,013 discloses a medical component holder formed as a rigid, elastic body that defines a channel receiving the medical component. One side of the channel is open and defines an opening that receives the medical component into the channel. Although the Bierman holder does not buckle during use, the holder is secured to the patient by adhesive patches that have the same disadvantages of adhesive tape.

Rogers et al. U.S. Pat. No. 5,555,881 discloses an endotracheal tube positioner that holds and positions an endotracheal tube placed into the trachea of a medical patient. The tube positioner includes a pair of hinged holder parts that can open and then close about the tube. One of the holder parts is attached to a lip bumper that extends beyond the opening. The construction of the Rogers et al. endotracheal tube positioner is not convenient for receiving and holding a catheter because of interference from the lip bumper.

Wright et al. U.S. Pat. No. 8,016,792 discloses a catheter securement device that includes a hinged base and cover that that can open and then close about the catheter. The base is attached to an adhesive pad that extends in all directions away from the base. The construction of the Rogers et al. catheter securement device can make receiving and holding a catheter from a tray or other flat surface difficult because of interference from the base and cover members and from the adhesive pad.

Miles U.S. Pat. No. 5,916,199 discloses a medical tubing holding device that includes a hinged base and cover that opens and closes about the tubing. The Miles device is not designed to receive and hold tubing from a tray or other flat surface.

Thus there is a need for an improved medical component securement device that does not buckle during use, does not require the use of adhesives, and enables receiving a medical component device resting on a tray or other flat surface without interference from the securement device itself.

### SUMMARY OF THE DISCLOSURE

Disclosed is a medical component holder and related securement device that does not buckle and does not adhere to the patient's skin during use.

A medical component securement device includes a strap and a medical component holder attached to the strap. The strap encircles a limb of a patient to secure the medical component holder to the patient. The strap includes fasteners that enable the strap to snuggly fit the patient.

In an embodiment, the strap includes holes that enable the medical component holder to be attached to the strap.

In a second embodiment, the strap includes strap portions extending from opposite sides of the medical component holder. In a variant embodiment, the strap portions include holes that enable one or more auxiliary medical component holders to be removably attached to the strap. An auxiliary medical component holder can be selectively positioned along the strap and can be selectively positioned on either side of the medical component holder.

In embodiments, the medical component holder is a unitary body having a channel that receives the medical component held by the body. The body may be a one-piece body, or may be made of multiple body members that can move between opened and closed states of the medical component holder.

In one particular embodiment, the medical component holder is designed to hold one of a catheter and a tubing extending from the catheter. The auxiliary medical component holder is designed to hold the other of the catheter and the tubing extending from the catheter.

The disclosed medical component securement device is easy to use in placing a medical component in the medical component holder or, if used, the auxiliary medical component holder. The disclosed medical securement device comfortably secures the medical component to the body without buckling. The strap enables the medical component to be adjustably secured to the patient without the discomfort of adhesives or tapes.

Other objects and features of the disclosure will become apparent as the description proceeds, especially when taken in conjunction with the accompanying drawing sheets illustrating one or more illustrative embodiments.

### BRIEF SUMMARY OF THE DRAWINGS

FIG. 1 is a perspective view of a first embodiment medical component securement device and an auxiliary medical component holder attached to the device.

FIG. 2 is a front view of the medical component holder of the device shown in FIG. 1 in an opened position.

FIG. 3 is a perspective view of a first body member of the medical component holder shown in FIG. 2.

FIGS. 4-6 are top, side, and back views respectively of the body member shown in FIG. 3.

FIG. 7 is a perspective view of a second body member of the medical component holder shown in FIG. 2.

FIGS. 8-10 are top, side, and back views respectively of the body member shown in FIG. 7.

FIG. 11 is a top view of a portion of a strap of the medical component securement device shown in FIG. 1.

FIG. 12 is similar to FIG. 1 but illustrates the medical component securement device holding a catheter and the auxiliary medical component holder holding the tubing extending from the catheter.

FIG. 13 is a bottom view of the medical component holder shown in FIG. 12 holding the catheter.

FIG. 14 is a vertical sectional view of the medical component holder in FIG. 12 holding the catheter.

FIG. 15 is a perspective view of the auxiliary medical component holder shown in FIG. 1.

FIGS. 16-18 are top, front, and side views of the auxiliary medical component holder shown in FIG. 15.

FIG. 19 illustrates a second embodiment medical component securement device.

### DETAILED DESCRIPTION

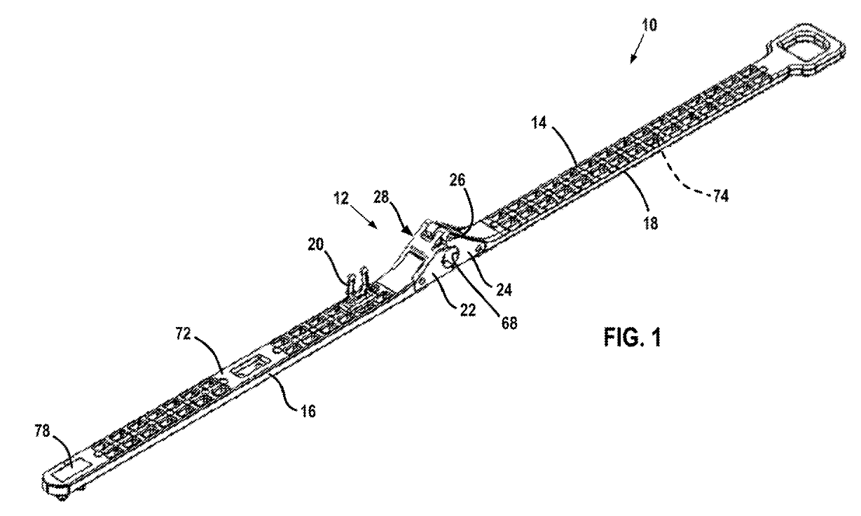

FIG. 1 illustrates a first embodiment medical component securement device 10. The medical securement device includes a medical component holder 12 attached to a strap 14 that secures the component holder 12 to the patient's body. The strap 14 is formed from a flexible, elongate first strap portion 16 secured to one side of the holder 12 and a flexible, elongate second strap portion 18 secured to the other side of the holder 12.

Also shown in FIG. 1 is a second, auxiliary medical component holder 20 that is a separate member from the securement device 10. The auxiliary medical component holder 20 is shown in FIG. 1 being removably attached to the strap 14 for holding a second medical component. The second medical component holder 20 will be described in more detail later below.

The medical component holder 12 is a unitary body (that is, a body not made of members that are intended to be separated from each other during use). The body is formed by a generally rigid first body member 22 and a generally rigid second body member 24. The body members 22, 24 are pivotally connected to one another by a hinge pin 26. The hinge pin defines the pivot axis. The body members 22, 24 although movable with respect to one another are not intended to be totally separable from one another during the lifetime of the medical component holder 12.

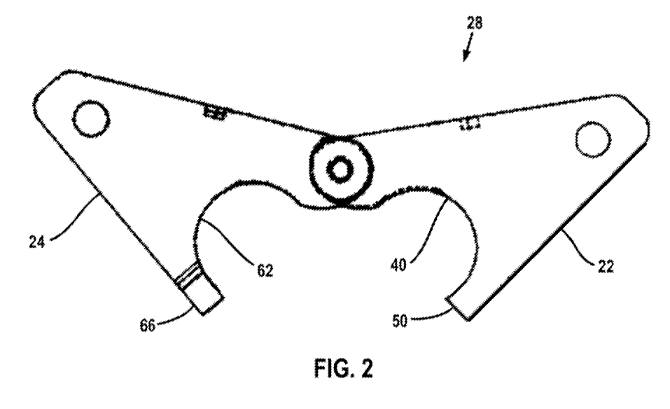

The body members 22, 24 are movable with respect to each other between an opened position shown in FIG. 2 and a closed position shown in FIG. 1. When the body members 22, 24 are in the closed position, the body members 22, 24 are fastened together and define a holder body 28. The fastening of the members 22, 24 resists movement of the body members 22, 24 from the closed position towards the opened position and enables the holder body 28 to function as a generally rigid body when holding a medical component.

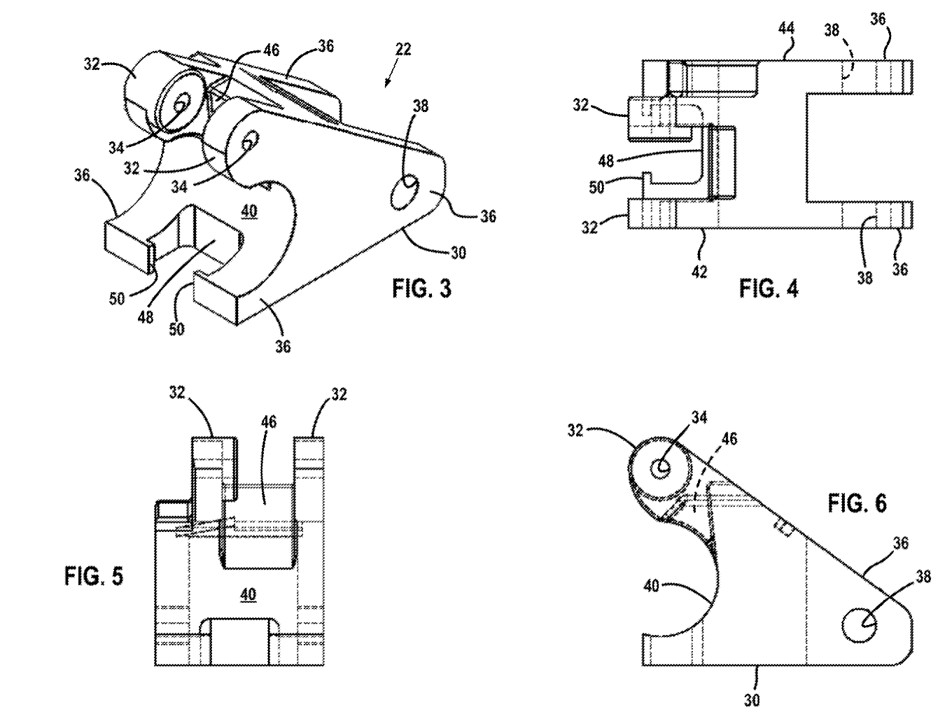

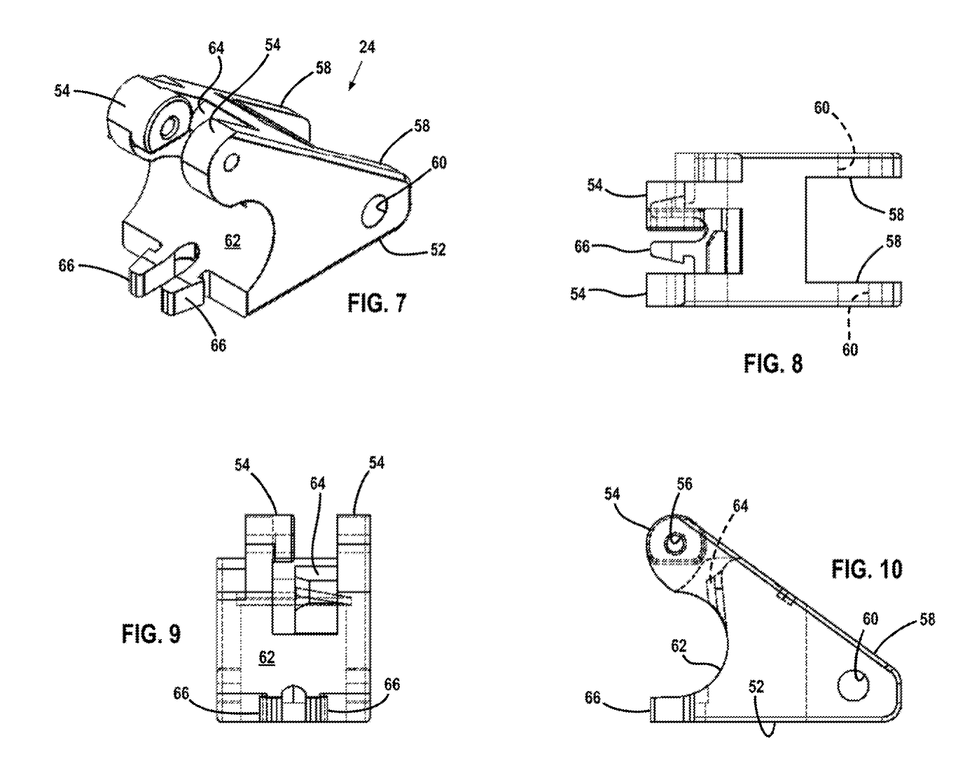

FIGS. 3-10 illustrate the first and second body members 22, 24.

The first body member 22 is a generally triangular-shaped member having a flat lower side 30 that lies against the patient's skin during use. A pair of hinge knuckles 32 are located at an upper side of the member and include co-axial hinge pin holes 34 that receive the hinge pin 26. On the right side of the member 22 as viewed in FIG. 6 are a pair of lugs 36 also located on the lower side 30 of the first body member. Co-axial strap pin holes 38 extend through the lugs 36 and receive a pin (not shown) attaching the strap portion 16 to the first body member 22.

On the left side of the member 22 as viewed in FIG. 6 is a curved, concave wall 40 that is generally semi-circular in cross-section. The wall 40 forms an outer wall of the body member 22 and extends the thickness of the member 22 between opposite lateral outer sides 42, 44 of the member 22.

The illustrated holder body 28 is intended to hold an intravenous catheter having a circular cylinder body. The radius of curvature of the wall 40 is selected to closely conform to the outer radius of the catheter to lightly grip the catheter when holding the catheter. The particular style of catheter to be held by the body 28 also includes a radially projecting lug extending from the outer surface of the catheter body. To accommodate the catheter lug, a slot 46 is formed in the first body member 22 that extends upwardly from the wall 40 into the body member 22. The walls facing the slot 46 will also assist in resisting relative rotation of the catheter when the catheter is held in the holder body 28.

Below the wall 40 on the lower side 30 is a female fastener member formed as a receptacle 48 open from the left side of the body member 22. A pair of fixed latches 50 define a narrowed opening of the receptacle. The receptacle 48 cooperates with a male fastener member on the second body member 24 to releasably fasten the body members 22, 24 together when forming the holder body 28.

The second body member 24 is similar to the first body member 22 and includes a flat lower side 52, a pair of hinge knuckles 54 on the upper side of the body member 24, coaxial hinge pin holes 56 extending through the knuckles that receive the hinge pin 26, a pair of lugs 58 on the right side of the member 24 as viewed in FIG. 10, the lugs 58 also located on the lower side 52, and co-axial strap pin holes 60 that extend through the lugs 58 and receive a pin (not shown) attaching the strap portion 18 to the second body member 24.

On the left side of the member 24 as viewed in FIG. 10 is a curved, concave wall 62 that is generally semi-circular in cross-section and has the same radius as the wall 40. The wall 62 forms an outer wall of the body member 24 and, like the wall 40, extends the thickness of the member 24. A slot 64 is formed in the second body member 24 that extends upwardly from the wall 62 into the body member 22. The slot 64 will face the slot 46 when the body members 22, 24 form the holder 28 to receive the lug of the catheter.

Below the wall 62 on the lower side 52 is a male fastener member formed as a pair of latch fingers 66 that extend away from the member 22. The latch fingers 66 are received into the receptacle 48 and move behind the latches 50 to engage the latches 50. The fastener members releasably buckle the body member 24 and the body member 22 together when the body members 22, 24 are in the closed position. The engaged latch fingers 66 and latches 50 resist opening of the closed holder 28, that is, the latch fingers and latches resist pivotal movement of the body members 22, 24 away from each other.

When the body members 22, 24 are buckled together in the closed position and form the holder 28, the walls 40, 46 face each other and define circular cylindrical channel 68 extending through the thickness of the body. See FIG. 1. The channel 68 extends along a longitudinal axis parallel the hinge pin 26 and is open at both longitudinal ends of the channel. The channel 68 is located nearer the lower flat sides 30, 52 than the upper sides of the body members.

In other possible embodiments the longitudinal axis of the channel 68 is not parallel with the pivot axis but is instead inclined with respect to the pivot axis so that the channel 68 would extend away from the pivot axis as the channel 68 extends from one open end of the channel to the other open end of the channel.

The slots 46, 64 face one another and cooperate to define a radial slot in the body holder 28 extending away from the channel 68.

FIG. 2 illustrates the body members 22, 24 in the opened position (the strap 14 is omitted from the drawing for clarity). The body members 22, 24 have each pivoted away from the closed position away from each other about 45 degrees about the pivot axis. The walls 40, 62 both face downwardly as viewed in FIG. 2.

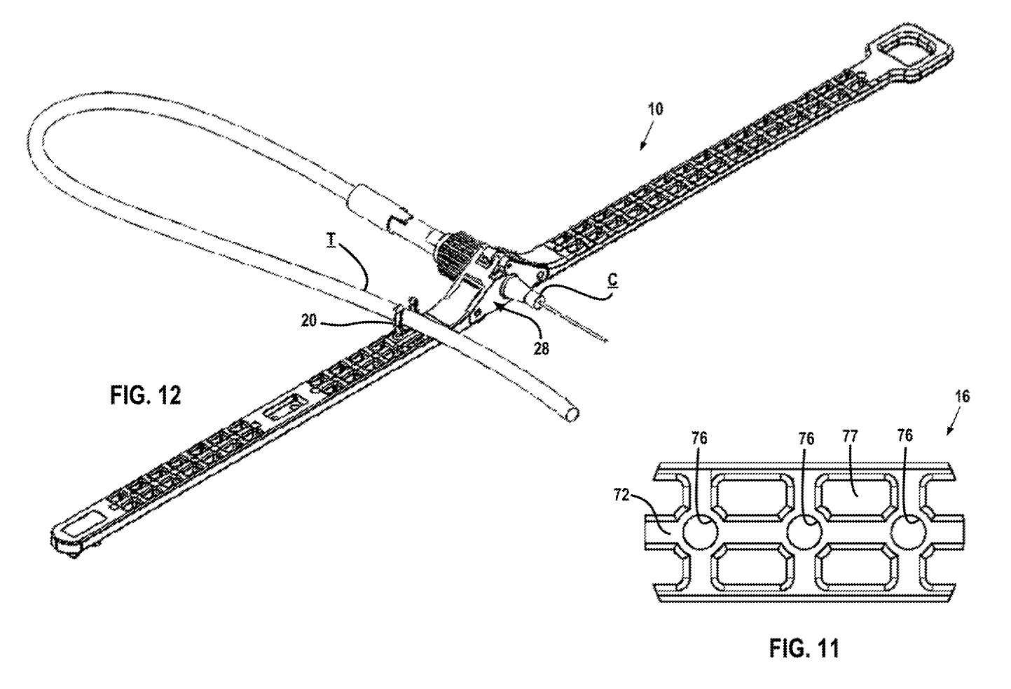

The strap portions 16, 18 are similar to one another, and FIG. 11 illustrates part of a strap portion. The strap portions are connected to the respective body members 22, 24 by strap pins extending through the lugs 36, 38 in a conventional manner used to attach straps to watch (timepiece) lugs. The strap portions 16, 18 are permanently attached to their respective body members, that is, except for replacement due to wear and the like, the strap portions 16, 18 are intended to remain attached to the body members during normal use of the securement device 10 in receiving, holding, and removing a medical component from the medical component holder 12.

Each strap portion has an outer side 72 that will be on the outside of the strap when the strap is fastened around the patient's limb and an opposite inner side 74 that will be on the inside of the strap and against the patient's skin. The lug strap holes 30, 60 are positioned such that the lower sides of the strap portions immediately adjacent to the holder 28 are essentially flush with the lower holder sides 30, 52 for patient comfort during use.

Each strap portion 16, 18 extends along its length dimension from the end attached to the strap pin to a free end. Uniformly spaced along the length of each strap portion are a number of like through holes 76. Each through-hole 76 is defined by an annular wall extending through the thickness of the strap portion and is open at both ends of the hole. The strap portions 16, 18 each include additional through-openings 77 for skin ventilation, increased strap flexibility, material savings, and the like.

In the illustrated embodiment, the holes 76 extend substantially the full length of each strap portion 16, 18. The two holes 76 of the strap portion 16 immediately adjacent to the free end receive a strap fastener 78 that includes a pair of studs that extend from the holes and are received in the appropriate pair of holes 76 of the strap portion 18 for adjustable, comfortable fit of the strap 14 around the limb of a patient.

The strap 14 may be made in various lengths for securing (for example) the holder body and any auxiliary component holders to the wrist, leg, torso, head, neck, or other portion of a patient. The strap 14 may be formed from plastic, nylon, rubber, silicone, or other materials used for conventional watch bands, or may be made from other suitable materials, including those suitable for molding or 3-D printing of the strap.

Embodiments of the strap may utilize different strap fasteners, such as hook and loop fasteners, buckles, or other conventional strap fasteners known in the prior art.

To place a medical component in the holder body 28, the first and second body members 22, 24 are moved to or near the opened position and the medical component is placed between the first and second body members. The channel walls 40, 62 are aligned with the medical component as needed for receiving the intended portion of the component within the channel. The medical component could also be placed in the recess defined by one of the channel walls 40, 62. The body members are then pivoted about the pivot axis towards each other to close the holder body 28 and receive the medical component in the channel between the two body members. The fasteners 46, 66 buckle the body members together in the closed position to complete the capture of the medical component in the component holder 28.

To release the medical component, the fasteners are unbuckled from one another, the component holder 28 is then opened, and the medical component removed from the component holder.

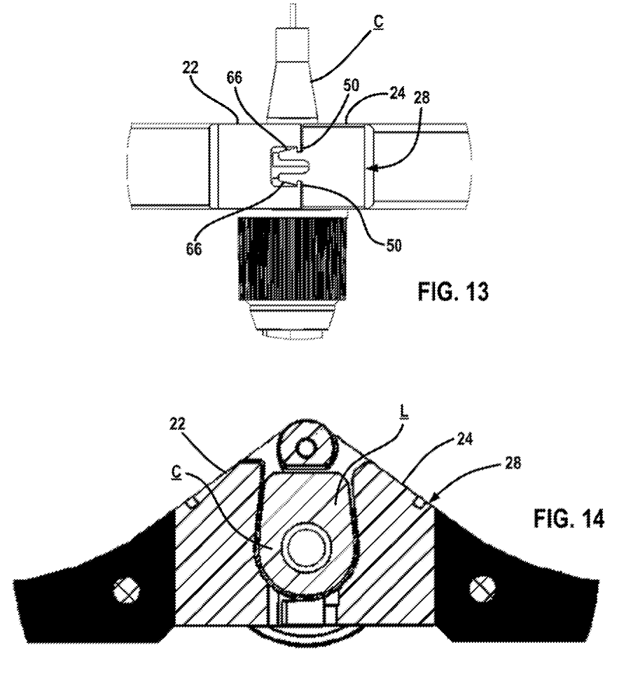

FIG. 12 illustrates the medical component securement device 10 holding both a catheter C in the component holder 28 and tubing T attached to the auxiliary component holder 20. FIG. 13 is a view of the bottom of the holder 28 showing engagement of the latch fingers 66 with the latches 50. FIG. 14 is a sectional view through the holder 28 showing the catheter lug L received inside the catheter holder 28. The holder 28 or the strap 14 may include visual and/or tactile indicia indicating the desired orientation of the medical component, the intended component size to be held in the holder, or other information.

The catheter C may be placed in the component holder 28 prior to catheter insertion in the vein, or the catheter may be placed in the component holder 28 after catheter insertion in the vein. The holder body 28 may perform the function of the catheter lug L to receive a force urging the catheter into the vein.

As demonstrated in FIGS. 1 and 12, the holes 76 of the strap 14 perform an auxiliary function in addition to permitting adjustable sizing of the strap 14. The holes 76 also enable fastening one or more auxiliary medical component holders, such as the second medical component holder 20, to the strap. The strap secures both the component holder 28 and any auxiliary medical component holders attached to the strap to the patient's body.

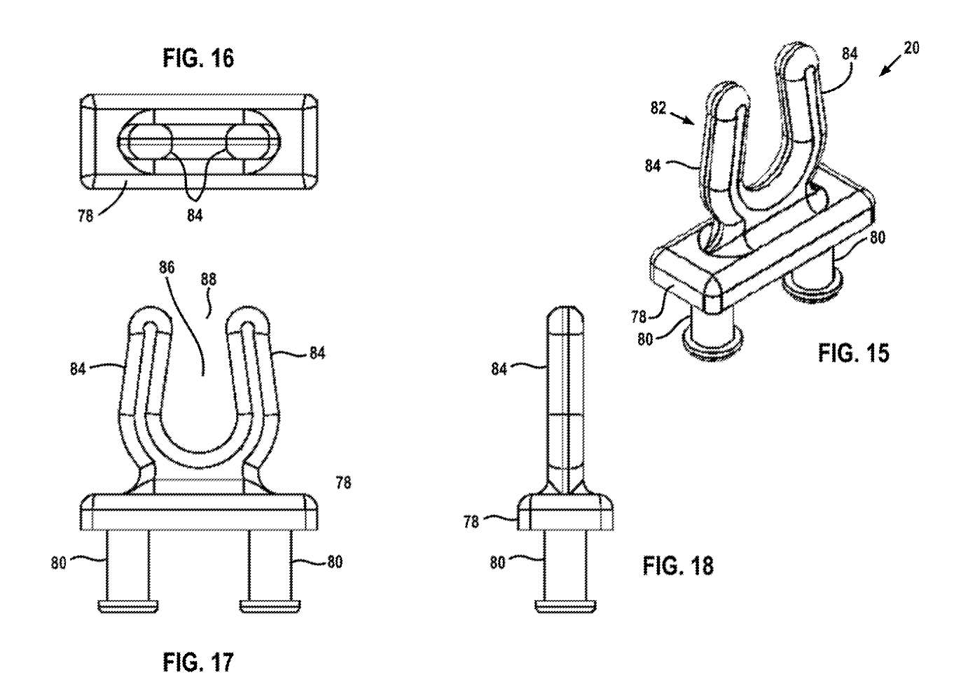

As shown in FIGS. 15-18, an auxiliary component holder formed as the holder 20 has a flat base 78, a pair of like studs 80 extending from one side of the base, and a medical component holder 82 on the other side of the base.

The illustrated component holder 82 is intended to hold the tubing connected to the catheter held by the holder body 28 as shown in FIG. 12. The component holder 82 includes a pair of prongs 84 that extend from the base 78 and define a gap 86 between the prongs. The prongs 84 are curved and define a narrowed opening 88 into the gap. The prongs 84 are elastically deformable to permit tubing to be received into the gap. The prongs 84 are intended to hold tubing of a specific outer diameter and the prongs 84 cooperate with the tubing to generate an interference fit that secures the tubing received in the gap 86 to the component holder 20.

The studs 80 are spaced apart and sized to be received from the top side of the strap 14 into a pair of the holes 76. The studs 80 may have radially enlarged ends as shown that engage the inside of the strap and resist detachment of the auxiliary medical component holder 20 from the strap.

Alternative embodiments of an auxiliary medical component holder may be designed to hold other types or sizes of medical components, and may be designed to hold a medical component in a different manner than that of the auxiliary medical component holder 20. For example, the component holder may be similar to the medical holder 12 but adapted for mounting on a base similar to the base 78 rather than being attached to a strap.

An auxiliary medical component holder such as the component holder 20 may be placed in any available pair of adjacent strap hole 76, thereby permitting the location of an auxiliary component holder along the strap 14 to be selected or changed as needed for optimal placement along the strap. The auxiliary medical component holder may be placed on either side of the holder body 28 as desired. Multiple auxiliary medical component holders may be simultaneously held by the strap, and those multiple auxiliary medical component holders do not necessarily have to be identical to one another.

An auxiliary medical component holder may be designed to hold the same type of medical component as does the strap holder 28, or may be designed to hold a different type or size of medical component, or may be designed to cooperate with the strap holder 28 in cooperatively holding the same medical component. Yet other embodiments of the auxiliary medical component holder may have only one stud 80 or more than two studs 80 for attachment of the auxiliary component holder to the strap 14.

A medical component may be placed in the medical component holder prior to the auxiliary medical component holder being attached to the strap, or after the auxiliary medical component holder is attached to the strap.

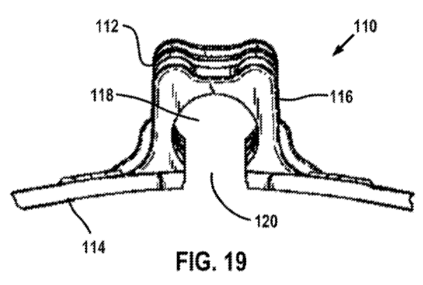

FIG. 19 illustrates a second medical component securement device 110. The medical securement device includes a medical component holder 112 attached to a strap 114 for securing the component holder 112 to the patient's body. The strap 114 is identical to the strap 14 and so only the medical component holder 112 will be described.

The medical component holder 112 is disclosed in Bierman U.S. Pat. No. 9,562,348. Bierman U.S. Pat. No. 9,562,348 is incorporated by reference into this application as if fully set forth herein.

The medical component holder 112 has a generally rigid, one member body 116 that defines a channel 118 extending through the body between open ends of the channel. An opening 120 extends along the bottom of the channel 118 and opens from the outside of the body 116 into the channel 118. The strap portions of the strap 114 are attached to opposite sides of the body 116.

A medical component is received in the channel 118 through the opening 120. The body 116 may have sufficient elasticity to enable the medical component to pass through the opening 120 and be received in the channel 118. The body 116 may form an elastic interference fit with the medical component held in the channel 118.

While one or more embodiments have been disclosed and described in detail, it is understood that this is capable of modification and that the scope of the disclosure is not limited to the precise details set forth but includes modifications obvious to a person of ordinary skill in possession of this disclosure, including (but not limited to) changes in material selection, size, operating ranges (temperature, volume, displacement, stroke length, concentration, and the like), environment of use, and also such changes and alterations as fall within the purview of the following claims.

What is claimed is:

1. A medical component securement device for holding and securing a medical component to a medical patient, the device comprising:
a medical component holder, a first elongate, flexible strap portion, and a second elongate, flexible strap portion,
the medical component holder being formed from a rigid first body member, a rigid second body member, and a hinge connecting the first and second body members with one another, the hinge defining a pivot axis, the first and second body members being pivotally movable about the pivot axis towards each other from an opened state of the medical component holder to a closed state of the medical component holder;
the medical component holder having opposite ends separated by a thickness of the medical component holder and first and second sides, the hinge being disposed adjacent to the first side;
when the medical component holder is in the closed state the first and second body members cooperatively define and at least partially surround a channel opening extending along a channel axis through the thickness of the medical component holder for receiving the medical component, the channel opening being disposed between the first and second sides of the medical component holder, each of the first and second body members having a surface disposed on the second side, the surfaces engageable with one another to thereby resist further pivotal movement of the body members towards each other from the opened state of the medical component holder;
when the medical component holder is in the opened position the surfaces are spaced apart from one another and the chamber opening is open to the second side of the medical component holder;
each of the first and second strap portions being attached to a respective one of the first and second body members;
each strap portion having a width dimension where the strap portion is attached to the respective body member and a length dimension being transverse to the width dimension, the width dimension extending substantially parallel with the hinge axis, the strap portion being extendable along the length dimension away from the respective body member; each strap portion being extendable initially away from the first side end of the medical component holder towards the second side of the medical component holder to a free end portion of the strap portion when the medical component holder is in the closed state with the free end portions of the strap portions being releasably fastenable to one another to form a strap disposed on the second side of the component holder adjacent away from the hinge;
the strap portions being extendable away from the second side of the medical component holder when the medical component holder is in the opened state the medical component can be placed between the first and second body members without interference from the strap portions.

2. The medical component holder of claim 1 wherein the hinge comprises a pivot pin defining the pivot axis and extending through each of the first and second body holders.

3. The medical component holder of claim 1 wherein the channel axis is parallel with the pivot axis.

4. The medical component holder of claim 1 wherein the channel opening has opposed open ends, the channel axis is not parallel with the pivot axis, and the channel opening extends away from the pivot axis as the channel opening extends from one open end to the other open end of the channel opening.

5. The medical component holder of claim 1 wherein the first and second body members comprise a male fastener and a female fastener, the male fastener being received in the female fastener and forming an interference fit with the female fastener when the medical component holder is in the closed state.

6. The medical component holder of claim 1 wherein the channel defined by the first and second body members when the medical component holder is in the closed state comprises a slot extending away from the chamber axis and into one or both of the first and second body members.

7. The medical component holder of claim 1 wherein the strap portions comprise fastening members that releasably fasten the strap portions together when forming the strap, the fastening members comprising buckles or hook-and-loop fasteners.

8. The medical component holder of claim 1 wherein the strap portions each include a plurality of spaced-apart through-holes extending along the lengths of the strap portions.

9. The medical component holder of claim 1 wherein an auxiliary component holder is held in one or more of the through-holes.

10. The medical component holder of claim 1 wherein the channel opening is configured to hold a catheter or tubing attached to a catheter.

11. The medical component holder of claim 1 wherein each of the first and second body members is generally triangular shaped.

12. The medical component holder of claim 11 wherein the hinge is disposed adjacent to and extending through a respective first corner of the triangles, the strap portions are each attached adjacent to a respective second corner of the triangles, and the surfaces are each disposed at or adjacent to a respective third corner of the triangles.

13. The medical component holder of claim 1 wherein each of the first and second body members has a flat outer surface disposed on the second end of the medical component holder.

14. The medical component holder of claim 1 wherein the first and second strap portions are pivotally mounted to the respective first and second body members.

15. The medical component holder of claim 14 wherein the first and second strap portions are connected to the first and second body members by pins.

16. The medical component holder of claim 1 wherein the strap portions do not extend beyond the second side of the medical component holder when the medical component holder is in the opened state.

17. The medical component holder of claim 1 wherein each of the first and second body members pivots about 45 degrees about the pivot axis when moving from the opened state to the closed state of the medical component holder.

18. The medical component holder of claim 1 wherein when the medical component holder is in the closed state the channel opening is closer to the second end than to the first end of the medical component holder.

19. The medical component holder of claim 1 wherein when the medical component holder is in the closed state the pivot axis and channel opening axis defines a first plane containing both axes and a second plane extending along the channel opening axis and perpendicular to the first plane, the pivot axis is on one side of the second plane, and where the first and second strap portions are attached to the first and second body members are each disposed on the one side of the second plane.

20. The medical component holder of claim 19 wherein the first and second body members are pivotally movable about the pivot axis from the closed state to the opened state of the medical component holder, and each of where the first and second strap portions are attached to the first and second body members moves away from the second plane as the first and second body members move from the closed state towards the open state of the medical component holder.

21. The medical component holder of claim 1 wherein when the medical component holder is in the closed state the pivot axis and the channel opening axis define a plane containing both axes, and where the first and second strap portions are attached to the respective first and second body members is located on opposite sides of the plane
